(19) United States
(12) Patent Application Publication
(10) Pub. No.: US 2023/0337695 A1
(43) Pub. Date: Oct. 26, 2023

(54) METHOD FOR PRODUCING CENTRE FILLED GUMMIES COMPRISING FILLINGS

(71) Applicant: ROUSSELOT BV, Gent (BE)

(72) Inventors: Ivo VERHEYE, Gent (BE); Claude CAPDEPON, Gent (BE); Jeff DAELMAN, Gent (BE)

(21) Appl. No.: 18/025,503

(22) PCT Filed: Oct. 29, 2021
(86) PCT No.: PCT/EP2021/080074

§ 371 Date: Mar. 9, 2023

(30) Foreign Application Priority Data
Oct. 30, 2020 (EP) 20204891.4
Nov. 20, 2020 (EP) 20208906.6

(51) Int. Cl. A23G 3/54; A23G 3/34; A23G 3/42; A23G 3/36; A23G 3/48; A61K 9/00; A61K 35/644; A61K 31/375; A61K 47/42; A61K 31/192; A61K 33/26

(52) U.S. Cl. CPC A23G 3/54; A23G 3/0072; A23G 3/42; A23G 3/368; A23G 3/48; A61K 9/0056; A61K 35/644; A61K 31/375; A61K 47/42; A61K 31/192; A61K 33/26

(57) ABSTRACT

The present invention provides a method for producing a seamless centre filled gummy; wherein the filling comprises at least one active ingredient; the method comprising the steps of: 1) preparing a gelatinous shell composition by a first gelatine product in water to form a first gelatine solution; 2) preparing a filling composition, wherein the filling composition comprises at least one bulking agent and one or more active ingredients dissolved or suspended in said filling composition; 3) depositing the gelatinous shell composition and the filling composition within the same timeframe; 4) setting the deposited gelatinous shell composition; wherein the temperature Tᵢₙₙₑᵣ of the filling composition upon deposition is at most 45° C., and wherein the viscosity of the filling composition upon deposition ranges between 500 and 30000 mPa·s. The invention further provides seamless centre filled gummy comprising an active ingredient, which may be produced by the inventive method.

### FIELD OF THE INVENTION

The invention relates to the field of confectionery, in particularly centre filled confectionery, more in particularly to centre filled confectionery wherein the filling comprises an active ingredient.

### BACKGROUND OF THE INVENTION

There is an ongoing search for new delivery forms for active ingredients such as for pharmaceutical and/or nutraceutical ingredients. Whereas capsules and tablets are the most popular delivery forms for nutraceutical and/or pharmaceutical ingredients, consumers typically prefer an easier way to swallow the dosage form. In this context gummies and chewable tablets are becoming more interesting due to their tasty and easy intake.

However, formulating and producing a gummy which comprises an active ingredient is not as straightforward, as some of the active ingredient interfere with commonly used ingredients for making gummies. This interference may lead to gelification problems. Even more, the production methods of gummies may not be compatible with all active ingredients, which may lead to decomposition of the active ingredient and/or variation in the dosage of the active ingredient in the end product.

When formulating active ingredients in centre filled gummies, leakage of the filling out of the centre filled gummy should be avoided, especially for liquid fillings, the shell around the filling should be leak-free. Preferably, the filling should be centred in the centre filled gummy. Often when co-depositing, the filling and the shell, the filling floats to the top of the mould, before the shell is set. Typically, a foam layer is applied on top such a gummy to seal of the filling. As this requires a third composition, e.g. the foam composition, and/or another production step, there is a demand to avoid having to apply the third composition all together.

### SUMMARY OF THE INVENTION

Described herein are methods to produce delivery forms, in particular centre filled gummies, allowing to facilitate uniform dosage of the active ingredient in the centre filled gummy preferably while at the same time avoiding the degradation of the active ingredient during manufacturing or storage. Preferably and advantageously, the methods avoid interaction between the gelatine and the active ingredient, e.g. Vitamin B8 (biotin), which leads to gelification problems.

The delivery forms such as centre filled gummies as described herein provide in soft products with a solid, pasty or liquid centre. Their filling boosts extended possibilities on type of active ingredient used and elevates the dosing efficiency as compared to standard functional confectionery.

The delivery forms such as centre filled gummies as described herein have a great taste, good look and are easy to swallow.

With this new delivery form, it is possible to bring a wide range of active ingredients to consumers, allowing them to enjoy a soft, chewy and tasty gummy, while taking their nutraceutical or pharmaceutical ingredients.

The delivery forms such as centre filled gummies as described herein are gelatine-based and enable starchless depositing during manufacturing.

One-shot depositing technology allows for more efficient manufacturing and provides products where the filling is completely embedded in the shell, without the need for two different shell compositions, or intermediate drying or setting times. It also allows for the production of a seamless centre filled gummy, which has a higher resistance to leaking of the filling from the centre filled gummy.

In a first aspect, the invention provides in a method for producing a seamless centre filled gummy, said seamless centre filled gummy comprising a gelatinous shell and a filling encased inside said gelatinous shell; wherein the filling comprises at least one active ingredient;
the method comprising the steps of:
   1) preparing a gelatinous shell composition by:
      1a) dissolving a first gelatine product in water to form a first gelatine solution;
      1b) optionally adding at least one first sweetener and optionally one or more secondary ingredients to the first gelatine solution;
   2) preparing a filling composition, wherein the filling composition comprises at least one bulking agent and one or more active ingredients dissolved or suspended in said filling composition;
   3) depositing the gelatinous shell composition and the filling composition within the same timeframe;
   thereby fully encasing the filling composition with the gelatinous shell composition;
   4) setting the deposited gelatinous shell composition;
wherein the temperature Tᵢₙₙₑᵣ of the filling composition upon deposition is at most 45° C., and wherein the viscosity of the filling composition upon deposition ranges between 500 and 30000 mPa·s, preferably between 2000 and 8000 mPa·s or between 1500 and 8000 mPa·s.

In an alternative aspect, the invention provides in a method for producing a seamless centre filled gummy, said seamless centre filled gummy comprising a gelatinous shell and a filling encased inside said gelatinous shell; wherein the filling comprises at least one active ingredient;
the method comprising the steps of:
   1) preparing a gelatinous shell composition by:
      1a) dissolving a first gelatine product in water to form a first gelatine solution;
      1b) optionally adding at least one first sweetener and optionally one or more secondary ingredients to the first gelatine solution;
   2) preparing a filling composition, wherein the filling composition comprises at least one bulking agent and one or more active ingredients dissolved or suspended in said filling composition;
   3) depositing the gelatinous shell composition through the outer nozzle at a temperature Tₒᵤₜₑᵣ, over a time interval t₁-t₄, starting at time t₁ and lasting until time t₄; and depositing the filling composition trough the inner nozzle at a temperature Tᵢₙₙₑᵣ, over a time interval t₂-t₃, starting at time t₂ and lasting until time t₃;
      wherein t₁<t₂<t₃<t₄;
   thereby fully encasing the filling composition with the gelatinous shell composition;
   4) setting the deposited gelatinous shell composition;
wherein the temperature Tᵢₙₙₑᵣ of the filling composition upon deposition is at most 45° C., and wherein the viscosity of the filling composition upon deposition ranges between 500 and 30000 mPa·s.

In some embodiments, the invention provides a method for producing a seamless centre filled gummy comprising the steps as defined above in aspects (i) and (ii), and wherein the temperature Tᵢₙₙₑᵣ of the filling composition upon deposition is at most 45° C., and wherein the viscosity of the filling composition upon deposition ranges between 2500 and 30000 mPa·s.

In some embodiments, the invention provides a method for producing a seamless centre filled gummy comprising the steps as defined above in aspects (i) and (ii), and wherein the temperature Tᵢₙₙₑᵣ of the filling composition upon deposition is at most 45° C., and wherein the viscosity of the filling composition upon deposition ranges between 3500 and 30000 mPa·s.

In some embodiments, the invention provides a method for producing a seamless centre filled gummy comprising the steps as defined above in aspects (i) and (ii), and wherein the temperature Tᵢₙₙₑᵣ of the filling composition upon deposition is at most 45° C., and wherein the viscosity of the filling composition upon deposition ranges between 4500 and 30000 mPa·s.

In some embodiments, said one or more active ingredients are selected from the list comprising vitamins, minerals, probiotics, herbal extracts, essential fatty acids, hydrolyzed collagen peptides, pharmaceuticals or a combination thereof.

In some embodiments, the temperature Tₒᵤₜₑᵣ of the gelatinous shell composition upon deposition is at least at least 40° C. to at most 100° C., preferably at least 45° C. to at most 85° C. preferably at least 50° C. to at most 75° C., preferably at least 55° C. to at most 65° C., preferably 60° C.; and/or wherein the temperature Tᵢₙₙₑᵣ of the filling composition upon deposition is at most 40° C., preferably at most 35° C., preferably at most 30° C., preferably at most 25° C., preferably at most 23° C., preferably at most 20° C., preferably at most 15° C., preferably at most 10° C., preferably at most 7° C., preferably at most 5° C.

In some embodiments, the viscosity of the gelatinous shell composition at temperature Tₒᵤₜₑᵣ is at least 500 mPa·s to at most 30000 mPa·s, preferably at least 1000 mPa·s to at most 25000 mPa·s, preferably at least 1500 mPa·s to at most 20000 mPa·s, preferably at least 2000 mPa·s to at most 17000 mPa·s, preferably at least 2500 mPa·s to at most 15000 mPa·s, preferably at least 3000 mPa·s to at most 12000 mPa·s; and/or,

wherein the viscosity of the filling composition at temperature Tᵢₙₙₑᵣ is at least 1000 mPa·s to at most 25000 mPa·s, preferably at least 1500 mPa·s to at most 20000 mPa·s, preferably at least 2000 mPa·s to at most 17000 mPa·s, preferably at least 2500 mPa·s to at most 15000 mPa·s, preferably at least 3000 mPa·s to at most 12000 mPa·s.

In some embodiments, the absolute density of the gelatinous shell composition at temperature Tₒᵤₜₑᵣ is at least 1.25 g/cm³ to at most 1.50 g/cm³, preferably at least 1.30 g/cm³ to at most 1.45 g/cm³, preferably at least 1.32 g/cm³ to at most 1.40 g/cm³, preferably at least 1.35 g/cm³ to at most 1.38 g/cm³; and/or, wherein the absolute density of the filling composition at temperature Tᵢₙₙₑᵣ is at least 1.25 g/cm³ to at most 1.50 g/cm³, preferably at least 1.30 g/cm³ to at most 1.45 g/cm³, preferably at least 1.32 g/cm³ to at most 1.40 g/cm³, preferably at least 1.35 g/cm³ to at most 1.38 g/cm³.

In some embodiments, the first gelatine product has a gel strength of at least 200 g to at most 300 g, preferably at least 205 g to at most 290 g, preferably at least 210 g to at most 285 g, preferably at least 210 g to at most 270 g, preferably at least 215 g to at most 265 g, preferably at least 215 g to at most 250 g, preferably at least 215 g to at most 240 g, preferably at least 215 g to at most 235 g, the gel strength being determined at 6.67%, 10° C., 17H according to the method described by the Gelatine Manufacturers of Europe (GME).

In some embodiments, the first gelatine product has a viscosity of at least 1.0 mPa·s to at most 10.0 mPa·s, preferably at least 2.0 mPa·s to at most 8.0 mPa·s, preferably at least at least 3.0 mPa·s to at most 6.0 mPa·s, preferably at least at least 3.2 mPa·s to at most 5.5 mPa·s, preferably at least 3.4 mPa·s to at most 5.0 mPa·s, preferably at least 4.2 mPa·s to at most 5.0 mPa·s, preferably at least 4.4 mPa·s to at most 5.0 mPa·s, preferably at least 4.4 mPa·s to at most 4.7 mPa·s the viscosity being determined at 6.67%, 60° C. according to the method described by the Gelatine Manufacturers of Europe (GME).

In some embodiments, the first gelatine product has a pH of at least 4.0 to at most 7.0, preferably at least 4.5 to at most 6.5, preferably at least at least 4.5 to at most 6.2, preferably at least at least 5.0 to at most 6.2, preferably at least 5.3 to at most 6.2, preferably at least 5.3 to at most 6.0, the pH being determined at 6.67%, 45° C. according to the method described by the Gelatine Manufacturers of Europe (GME).

In some embodiments, the gelatinous shell composition and the filling composition are deposited in a non-starch mould, preferably a plastic mould, silicon mould, metal mould or a blister.

In some embodiments, the bulking agent is selected from the list comprising:
a second gelatine product;
food oil, such as coconut oil;
emulsified food oil;
collagen peptides or hydrolyzed collagen peptides;
soluble fibers, such as inulin, polydextrose;
carbohydrate polymers; such as dextrin, maltodextrin;
glycerol; or,
polyethylene glycol.

In some embodiments, the gelatinous shell composition comprises at least 10 weight % to at most 30 weight %, preferably at least 15 weight % to at most 25 weight %, preferably at least 18 weight % to at most 22 weight %, preferably about 20 weight % water; compared to the total weight of the gelatinous shell composition.

In a second aspect the invention provides in a seamless centre filled gummy, produced by a method according to an embodiment described herein.

In a third aspects, the invention provides in a seamless centre filled gummy comprising:
a gelatinous shell; and,
a filling encased inside said gelatinous shell;
wherein the gelatinous shell comprises:
a first gelatine product;
optionally at least one first sweetener; and,
optionally one or more secondary ingredients;
wherein the filling comprises at least one active ingredient and at least one ingredient selected from the following:
a. a second gelatine product;
b. food oil, such as coconut oil
c. emulsified food oil;
d. collagen peptides or hydrolyzed collagen peptides;
e. soluble fibers, such as inulin or polydextrose;
f. maltodextrin or dextrine;
g. glycerol; or,
h. polyethylene glycol.

In some embodiments, the first gelatine product and/or optionally the second gelatine product have a gel strength of at least 200 g to at most 300 g, preferably at least 205 g to at most 290 g, preferably at least 210 g to at most 270 g, preferably at least 210 g to at most 285 g, preferably at least 215 g to at most 265 g, preferably at least 215 g to at most 250 g, preferably at least 215 g to at most 240 g, preferably at least 215 g to at most 235 g, the gel strength being determined at 6.67%, 10° C., 17H according to the method described by the Gelatine Manufacturers of Europe (GME).

In some embodiments, the first gelatine product and/or optionally the second gelatine product have a viscosity of at least 1.0 mPa·s to at most 10.0 mPa·s, preferably at least 2.0 mPa·s to at most 8.0 mPa·s, preferably at least at least 3.0 mPa·s to at most 6.0 mPa·s, preferably at least at least 3.2 mPa·s to at most 5.5 mPa·s, preferably at least 3.4 mPa·s to at most 5.0 mPa·s, preferably at least 4.2 mPa·s to at most 5.0 mPa·s, preferably at least 4.4 mPa·s to at most 5.0 mPa·s, preferably at least 4.4 mPa·s to at most 4.7 mPa·s the viscosity being determined at 6.67%, 60° C. according to the method described by the Gelatine Manufacturers of Europe (GME).

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous. Embodiments of one aspect of the invention are embodiments of the other aspects of the invention unless clearly indicated to the contrary.

### DETAILED DESCRIPTION OF THE INVENTION

Before the present method and products of the invention are described, it is to be understood that this invention is not limited to particular methods, components, products or combinations described, as such methods, components, products and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention.

It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any ≥3, ≥4, ≥5, ≥6 or ≥7 etc. of said members, and up to all said members.

All references cited in the present specification are hereby incorporated by reference in their entirety. In particular, the teachings of all references herein specifically referred to are incorporated by reference. Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

The invention provides in a method for producing a seamless centre filled gummy, said seamless centre filled gummy comprising a gelatinous shell and a filling encased inside said gelatinous shell; wherein the filling preferably comprises at least one active ingredient;
the method comprising the steps of:
   1) preparing a gelatinous shell composition by:
      1a) dissolving a first gelatine product in water to form a first gelatine solution;
      1b) optionally adding at least one first sweetener and optionally one or more secondary ingredients to the first gelatine solution;
   2) preparing a filling composition, wherein the filling composition comprises at least one bulking agent and preferably one or more active ingredients dissolved or suspended in said filling composition;
   3) depositing the gelatinous shell composition and the filling composition, preferably within the same timeframe;
   thereby fully encasing the filling composition with the gelatinous shell composition;
   4) setting the deposited gelatinous shell composition;
      preferably wherein the temperature Tᵢₙₙₑᵣ of the filling composition trough the inner nozzle is at most 45° C., preferably at most 40° C. or 35° C. or 30° C., most preferably at most 25° C. or 23° C., or 20° C. or 10° C. or 7° C. or 5° C.; and,
      preferably wherein the viscosity of the filling composition upon deposition ranges between 500 and 30000 mPa·s or between 2000 and 12000 mPa·s, more preferably wherein the viscosity of the filling composition upon deposition ranges between 1500 and 30000 mPa·s, between 2000 and 30000 m Pa·s, or between 2500 and 30000 mPa·s, most preferably wherein the viscosity of the filling composition upon deposition ranges between 4700 and 30000 mPa·s or between 4500 and 30000 mPa·s or between 5000 and 30000 mPa·s.

In some embodiments, the temperature Tᵢₙₙₑᵣ is at most 40° C., preferably at most 35° C., preferably at most 30° C., preferably at most 25° C., preferably at most 23° C., preferably at most 20° C., preferably at most 15° C., preferably at most 10° C., preferably at most 7° C., preferably at most 5° C.

As used herein, the term “gummy” refers to resilient, soft, gelatin containing candies. Examples of well-known gummies are gummy bears (Haribo, Germany) and jelly babies (Bassetts, UK). As such gummy candies usually have a fruit taste, they are also known as fruit gum candy. Gummies are often prepared by casting a liquid candy composition comprising gelatine, optionally a bulking agent and water in a mold, whereafter the liquid composition is allowed to set to provide the gummy candies, which are subsequently removed from the mould.

As used herein, the term “centre filled gummy” refers to a gummy, which comprises an outer shell which encases a filling. Said filling can be liquid, pasty or solid, or even a mixture thereof.

As used herein, the term “seamless centre filled gummy” refers to a centre filled gummy, wherein no seam can be seen anywhere in the shell. Often this is achieved by depositing the shell in one continuous deposit. This is in contrast to seamed-centre filled gummies, wherein first a part of the shell is deposited, and set, followed by the addition of the filling, where over the second half of the shell is deposited, before the second half is being set. Such a multi-step process will leave a visible line or seam where the first half of the shell meets the second half of the shell.

As used herein the term “gelatinous shell composition” refers to a composition that can be deposited in a mould. Hence, the term refers to the material which forms the gelatinous shell before it has set. As used herein, the term “gelatinous shell” refers to the gelatinous shell composition after it has set.

As used herein the term “filling composition” refers to a composition that can be deposited in a mould. The “filling composition” may set into a “filling”, but may also remain unchanged during setting of the gelatinous shell. Therefore, the term “filling” is used to refer to the filling composition after the gelatinous shell has set.

As used herein the term “bulking agent” refers to a non-nutritive additive that increases the bulk (volume or weight) of a food without affecting its taste and keeping its utility and functionality intact.

The low temperatures Tᵢₙₙₑᵣ used in the method, have the advantage that setting of the gelatinous shell composition may be sped-up. The filling which may be deposited at a lower temperature Tᵢₙₙₑᵣ, then the temperature at which the gelatinous shell composition is deposited, may cool down the gelatinous shell composition faster after deposition, thereby increasing the gelation or setting of the gelatinous shell. A fast gelation or setting of the gelatinous shell composition may avoid mixing of the gelatinous shell composition and filling composition after depositing. Especially, when the active ingredient interferes with the gelatine or another ingredient of the gelatinous shell composition, this may be an advantage as this may provide a better gelation or less decomposition of the active ingredient. A fast gelation or setting of the gelatinous shell composition may also avoid movement of the filling during gelation or setting. Said movement can be caused by difference in density, fast gelation or setting may stop said movement. The low temperatures Tᵢₙₙₑᵣ may allow the production of centre filled gummies with a liquid filling, and this without the need of a liquifying agent.

The low temperature Tᵢₙₙₑᵣ used in the method may also avoid thermal degradation of the active ingredient. By avoiding thermal degradation, a more controlled dosing of the active ingredient may be facilitated. Non limiting examples of active ingredient that suffer from thermal degradation are vitamin C, and probiotics, such as L. acidophilus, L. plantarum, B. bifidum, . . . .

The method may allow for a large amount of different active ingredients to be formulated in the centre filled gummy, for example up to 10 vitamins. This may lead to a highly nutritious centre filled gummy. The viscosity of the filling composition at depositing is in the range that the centre fill gummy does not collapse on itself, before the gelatinous shell had the time to set. It may avoid the movement of the filling before the gelatinous shell had the time to set.

In some embodiments, the gelatinous shell composition and the filling composition are deposited in a mould through a concentric nozzle, said concentric nozzle comprising an outer nozzle which is arranged around an inner nozzle.

As used herein the term “within the same timeframe” refers to single continuous time interval (preferably t1-t4), wherein both the gelatinous shell composition and the filling composition are deposited, however a phase shift between the two depositions may be present.

In some embodiments, the gelatinous shell composition is deposited through the outer nozzle at a temperature Tₒᵤₜₑᵣ, over a time interval t₁-t₄, starting at time t₁ and lasting until time t₄; and,
the filling composition is deposited trough the inner nozzle at a temperature Tᵢₙₙₑᵣ, over a time interval t₂-t₃, starting at time t₂ and lasting until time t₃;
   wherein t₁<t₂<t₃<t₄.

The notation “t₁<t₂<t₃<t₄”, means that t₁ is chronological before t₂, which is chronological before t₃, which is chronologic before t₄. Hence, the deposition of the gelatinous shell composition starts before the deposition of the filling composition, and last longer than the deposition of the filling composition. This way, the filling composition is fully encapsulated by the gelatinous shell composition when depositing is over.

In some embodiments, the invention thus provides in a method for producing a seamless centre filled gummy, said seamless centre filled gummy comprising a gelatinous shell and a filling encased inside said gelatinous shell; wherein the filling comprises at least one active ingredient;
the method comprising the steps of:
   1) preparing a gelatinous shell composition by:
      1a) dissolving a first gelatine product in water to form a first gelatine solution;
      1b) optionally adding at least one first sweetener and optionally one or more secondary ingredients to the first gelatine solution;
   2) preparing a filling composition, wherein the filling composition comprises at least one bulking agent and one or more active ingredients dissolved or suspended in said filling composition;
   3) depositing the gelatinous shell composition through the outer nozzle at a temperature Tₒᵤₜₑᵣ, over a time interval t₁-t₄, starting at time t₁ and lasting until time t₄; and depositing the filling composition trough the inner nozzle at a temperature Tᵢₙₙₑᵣ, over a time interval t₂-t₃, starting at time t₂ and lasting until time t₃;
      wherein t₁<t₂<t₃<t₄;
   thereby fully encasing the filling composition with the gelatinous shell composition;
   4) setting the deposited gelatinous shell composition;
wherein the temperature Tᵢₙₙₑᵣ of the filling composition upon deposition is at most 45° C., preferably wherein the temperature Tᵢₙₙₑᵣ of the filling composition trough the inner nozzle is at most 45° C., preferably at most 40° C. or 35° C. or 30° C., most preferably at most 25° C. or 23° C., or 20° C. or 10° C. or 7° C. or 5° C.; and, preferably wherein the viscosity of the filling composition upon deposition ranges between 500 and 30000 mPa·s or between 2000 and 12000 mPa·s, more preferably wherein the viscosity of the filling composition upon deposition ranges between 1500 and 30000 mPa·s, or between 2500 and 30000 mPa·s, most preferably wherein the viscosity of the filling composition upon deposition ranges between 4700 and 30000 mPa·s or between 4500 and 30000 mPa·s or between 5000 and 30000 mPa·s.

In some embodiments, a phase offset is present between the depositing of the gelatinous shell composition and the filling composition.

As used herein the term “active ingredient” refers to a pharmaceutical and/or nutraceutical ingredients. A pharmaceutical ingredient may refer to a substance in a pharmaceutical formulation that is biologically active and is meant to produce the desired effect in the body. A nutraceutical ingredient or a ‘bioceutical’ may be a pharmaceutical alternative which claims physiological benefits. Preferably, nutraceutical ingredients should be interpreted as the US Food and Drug Administration (FDA) interprets the term. The active ingredient may be sold over-the-counter or a on prescription. The active ingredient may be a class II, class III or a class IV API; preferably a class II or a class IV API; most preferably a class II API according to the biopharmaceutics classification system. In some embodiments, the active ingredient is selected from the list comprising vitamins, minerals, probiotics, herbal extracts, essential fatty acids, hydrolyzed collagen peptides, pharmaceuticals or a combination thereof. In some embodiments, the active ingredient is a pharmaceutical active ingredient suitable for pain relief and/or anti-inflammation.

In some embodiments, the temperature Tₒᵤₜₑᵣ of the gelatinous shell composition upon deposition is at least 40° C. to at most 100° C., preferably at least 45° C. to at most 85° C., preferably at least 50° C. to at most 75° C., preferably at least 55° C. to at most 65° C., preferably 60° C. Such temperatures may allow the melt of the first gelatine product allowing the gelatinous shell composition to be deposited in a liquid form.

In some embodiments, the temperature Tₒᵤₜₑᵣ of the gelatinous shell composition upon deposition is at least 40° C., preferably at least 45° C., preferably at least 50° C., preferably at least 55° C., preferably 60° C. In some embodiments, the temperature Tₒᵤₜₑᵣ of the gelatinous shell composition upon deposition is at most 100° C., preferably at most 85° C., preferably at most 75° C., preferably at most 65° C., preferably 60° C. In some embodiments, the difference between and upon deposition is at least 5° C., preferably at least 7° C., preferably at least 10° C., preferably at least 15° C., preferably at least 20° C., preferably at least 25° C., preferably at least 30° C., preferably at least 40° C., preferably at least 50° C. Preferably, Tᵢₙₙₑᵣ is lower than Tₒᵤₜₑᵣ. The difference in temperature between the gelatinous shell composition and the filling composition upon deposition, results in a faster cooling of the hottest composition, so that gelation of the hottest composition upon depositing goes faster.

In some embodiments, the viscosity of the gelatinous shell composition at temperature Tₒᵤₜₑᵣ is at least 500 mPa·s to at most 30000 mPa·s, preferably at least 1000 mPa·s to at most 25000 mPa·s, preferably at least 1500 mPa·s to at most 20000 mPa·s, preferably at least 2000 mPa·s to at most 12000 mPa·s, preferably at least 2000 mPa·s to at most 17000 mPa·s, preferably at least 2500 mPa·s to at most 15000 mPa·s, preferably at least 3000 mPa·s to at most 12000 mPa·s.

In some embodiments, the viscosity of the gelatinous shell composition at temperature Tₒᵤₜₑᵣ is at least 500 mPa·s, preferably at least 1000 mPa·s, preferably at least 1500 mPa·s, preferably at least 2000 mPa·s, preferably at least 2500 mPa·s, preferably at least 3000 mPa·s.

In some embodiments, the viscosity of the gelatinous shell composition at temperature Tₒᵤₜₑᵣ is at most 30000 mPa·s, preferably at most 25000 mPa·s, preferably at most 20000 mPa·s, preferably at most 17000 mPa·s, preferably at most 12000 mPa·s, preferably at most 15000 mPa·s, preferably at most 12000 mPa·s. In some embodiments, the viscosity of the filling composition at temperature Tᵢₙₙₑᵣ is at least at least 1000 mPa·s to at most 25000 mPa·s, preferably at least 1500 mPa·s to at most 20000 mPa·s, preferably at least 2000 mPa·s to at most 12000 mPa·s, preferably at least 2000 to at most 17000 mPa·s, preferably at least 2500 mPa·s to at most 15000 mPa·s, preferably at least 3000 mPa·s to at most 12000 mPa·s.

In some embodiments, the viscosity of the filling composition at temperature Tᵢₙₙₑᵣ is at least 1000 mPa·s, preferably at least 1500 mPa·s, preferably at least 2000 mPa·s, preferably at least 2500 mPa·s, preferably at least 3000 mPa·s.

In some embodiments, the viscosity of the filling composition at temperature Tᵢₙₙₑᵣ is at most 25000 mPa·s, preferably at most 20000 mPa·s, preferably at most 17000 mPa·s, preferably at most 15000 mPa·s, preferably at most 12000 mPa·s.

In some embodiments, the viscosity of the gelatinous shell composition at temperature Tₒᵤₜₑᵣ and the viscosity of the filling composition at temperature Tᵢₙₙₑᵣ, do not differ more than 10000 mPa·s, preferably not more than 7000 mPa·s, preferably not more than 5000 mPa·s, preferably not more than 4000 mPa·s, preferably not more than 3000 mPa·s, preferably not more than 2000 mPa·s. In some embodiments, the viscosity of the gelatinous shell composition at temperature Tₒᵤₜₑᵣ and the viscosity of the filling composition at temperature Tᵢₙₙₑᵣ are about the same.

In some embodiments, when the viscosity of the gelatinous shell composition at a temperature Tₒᵤₜₑᵣ of 60° C. is about 8000 mPa·s, the viscosity of the filling composition at temperature Tᵢₙₙₑᵣ may range between 2000 mPa·s and 12000 mPa·s.

In some embodiments, when the viscosity of the gelatinous shell composition at a temperature Tₒᵤₜₑᵣ of 80° C. is about 5000 mPa·s, the viscosity of the filling composition at temperature Tᵢₙₙₑᵣ may range between 1500 mPa·s and 8000 mPa·s.

In some embodiments, the absolute density of the gelatinous shell composition at temperature Tₒᵤₜₑᵣ is at least 1.25 g/cm³ to at most 1.50 g/cm³, preferably at least 1.30 g/cm³ to at most 1.45 g/cm³, preferably at least 1.32 g/cm³ to at most 1.40 g/cm³, preferably at least 1.35 g/cm³ to at most 1.38 g/cm³.

In some embodiments, the absolute density of the gelatinous shell composition at temperature Tₒᵤₜₑᵣ is at least 1.25 g/cm³, preferably at least 1.30 g/cm³, preferably at least 1.32 g/cm³, preferably at least 1.35 g/cm³.

In some embodiments, the absolute density of the gelatinous shell composition at temperature Tₒᵤₜₑᵣ is at most 1.50 g/cm³, preferably at most 1.45 g/cm³, preferably at most 1.40 g/cm³, preferably at most 1.38 g/cm³.

In some embodiments, the absolute density of the filling composition at temperature Tᵢₙₙₑᵣ is at least 1.25 g/cm³ to at most 1.50 g/cm³, preferably at least 1.30 g/cm³ to at most 1.45 g/cm³, preferably at least 1.32 g/cm³ to at most 1.40 g/cm³, preferably at least 1.35 g/cm³ to at most 1.38 g/cm³.

In some embodiments, the absolute density of the filling composition at temperature Tᵢₙₙₑᵣ is at least 1.25 g/cm³, preferably at least 1.30 g/cm³, preferably at least 1.32 g/cm³, preferably at least 1.35 g/cm³.

In some embodiments, the absolute density of the filling composition at temperature Tᵢₙₙₑᵣ is at most 1.50 g/cm³, preferably at most 1.45 g/cm³, preferably at most 1.40 g/cm³, preferably at most 1.38 g/cm³.

In some embodiments, the absolute density of the gelatinous shell composition at temperature Tₒᵤₜₑᵣ and the absolute density of the filling composition at temperature Tᵢₙₙₑᵣ, do not differ more than 0.25 g/cm³, preferably not more than 0.20 g/cm³, preferably not more than 0.15 g/cm³, preferably not more than 0.10 g/cm³, preferably not more than 0.05 g/cm³, preferably not more than 0.02 g/cm³. Such a difference avoids movement of the filling before the gelatinous shell composition has set after depositing.

In some embodiments, the first gelatine product and/or the second gelatine product has a gel strength of at least 200 g to at most 300 g, preferably at least 205 g to at most 290 g, preferably at least 210 g to at most 285 g, preferably at least 210 g to at most 270 g, preferably at least 215 g to at most 265 g, preferably at least 215 g to at most 250 g, preferably at least 215 g to at most 240 g, preferably at least 215 g to at most 235 g, the gel strength being determined at 6.67%, 10° C., 17H according to the method described by the Gelatine Manufacturers of Europe (GME). Such gel strengths may result in a fast gelation of the gelatinous shell composition.

In some embodiments, the first gelatine product and/or the second gelatine product has a gel strength of at least 200 g, preferably at least 205 g, preferably at least 210 g, preferably at least 215 g, preferably at least 220 g, preferably at least 225 g, the gel strength being determined at 6.67%, 10° C., 17H according to the method described by the Gelatine Manufacturers of Europe (GME).

In some embodiments, the first gelatine product and/or the second gelatine product has a gel strength of at most 300 g, preferably at most 290 g, preferably at most 285 g, preferably at most 270 g, preferably at most 265 g, preferably at most 250 g, preferably at most 240 g, preferably at most 235 g, the gel strength being determined at 6.67%, 10° C., 17H according to the method described by the Gelatine Manufacturers of Europe (GME).

In some embodiments, the first gelatine product and/or the second gelatine product has a viscosity of at least 1.0 mPa·s to at most 10.0 mPa·s, preferably at least 2.0 mPa·s to at most 8.0 mPa·s, preferably at least 3.0 mPa·s to at most 6.0 mPa·s, preferably at least 3.2 mPa·s to at most 5.5 mPa·s, preferably at least 3.4 mPa·s to at most 5.0 mPa·s, preferably at least 4.2 mPa·s to at most 5.0 mPa·s, preferably at least 4.4 mPa·s to at most 5.0 mPa·s, preferably at least 4.4 mPa·s to at most 4.7 mPa·s; the viscosity being determined at 6.67%, 60° C. according to the method described by the Gelatine Manufacturers of Europe (GME).

In some embodiments, the first gelatine product and/or the second gelatine product has a viscosity of at least 1.0 mPa·s, preferably at least 2.0 mPa·s, preferably at least 3.0 mPa·s, preferably at least 3.2 mPa·s t, preferably at least 3.4 mPa·s, preferably at least 4.2 mPa·s, preferably at least 4.4 mPa·s, preferably at least 4.4 mPa·s; the viscosity being determined at 6.67%, 60° C. according to the method described by the Gelatine Manufacturers of Europe (GME).

In some embodiments, the first gelatine product and/or the second gelatine product has a viscosity of at most 10.0 mPa·s, preferably at most 8.0 mPa·s, preferably at most 6.0 mPa·s, preferably at most 5.5 mPa·s, preferably at most 5.0 mPa·s, preferably at most 5.0 mPa·s, preferably at most 5.0 mPa·s, preferably at most 4.7 mPa·s; the viscosity being determined at 6.67%, 60° C. according to the method described by the Gelatine Manufacturers of Europe (GME).

It has been found that a gelatin product with such high viscosities and such high gel strengths, may result in improved setting of the gelatin, which may be ideally suited for one shot technology, which result in a seamless centre filled gummy.

In some embodiments, the first gelatine product and/or the second gelatine product has a pH of at least 4.0 to at most 7.0, preferably at least 4.5 to at most 6.5, preferably at least 4.5 to at most 6.2, preferably at least 5.0 to at most 6.2, preferably at least 5.3 to at most 6.2, preferably at least 5.3 to at most 6.0, the pH being determined at 6.67%, 45° C. according to the method described by the Gelatine Manufacturers of Europe (GME).

In some embodiments, the first gelatine product and/or the second gelatine product has a pH of at least 4.0, preferably at least 4.5, preferably at least 4.5, preferably at least 5.0, preferably at least 5.3, preferably at least 5.3, the pH being determined at 6.67%, 45° C. according to the method described by the Gelatine Manufacturers of Europe (GME).

In some embodiments, the first gelatine product and/or the second gelatine product has a pH of at most 7.0, preferably at most 6.5, preferably most 6.2, preferably at most 6.2, preferably at most 6.2, preferably at most 6.0, the pH being determined at 6.67%, 45° C. according to the method described by the Gelatine Manufacturers of Europe (GME).

In some embodiments, the gelatinous shell composition and the filling composition are deposited in a non-starch mould, preferably a plastic mould, silicon mould metal mould or a blister. This may avoid the need to use starch. Starch has the disadvantage that is needs to be dried before it can be reused, and that it gets contaminated with the ingredients used in the gummy. Especially when an active ingredient is present in the gummy, the contamination of the starch may be problematic.

In some embodiments, the mould is made from metal, plastics, like silicone or polytetrafluorethylene (PTFE), or plastics coated with such a water repellant material.

In some embodiments, the bulking agent, preferably the bulking agent of the filling composition, is selected from the list comprising:
a second gelatine product;
food oil, such as coconut oil;
emulsified food oil;
collagen peptides or hydrolyzed collagen peptides;
soluble fibers, such as inulin, polydextrose;
carbohydrate polymers; such as dextrin, maltodextrin;
glycerol; or,
polyethylene glycol.

In some embodiments, the gelatinous shell composition comprises a bulking agent.

In some embodiments, the gelatinous shell composition comprises at least 10 weight % to at most 30 weight %, preferably at least 15 weight % to at most 25 weight %, preferably at least 18 weight % to at most 22 weight %, preferably about 20 weight % water; compared to the total weight of the gelatinous shell composition. Such low amount of water does not require or require only very limited amount of water to leave the composition, for the composition set or gelate. Classically, water may leave the composition trough evaporation or being drawn away by a drying agent, such as starch, which may be present in the mould.

In some embodiments, the gelatinous shell composition comprises:
at least 10 weight % to at most 30 weight %, preferably at least 15 weight % to at most 25 weight %, preferably at least 18 weight % to at most 22 weight %, preferably about 20 weight % water; compared to the total weight of the gelatinous shell composition; and,
a first gelatine product with a gel strength of at least 200 g to at most 300 g, preferably at least 205 g to at most 290 g, preferably at least 210 g to at most 285 g, preferably at least 215 g to at most 270 g, preferably at least 215 g to at most 265 g, preferably at least 215 g to at most 250 g, preferably at least 215 g to at most 240 g, preferably at least 215 g to at most 235 g, the gel strength being determined at 6.67%, 10° C., 17H according to the method described by the Gelatine Manufacturers of Europe (GME).

In some embodiments, the gelatinous shell composition comprises:
at least 10 weight % to at most 30 weight %, preferably at least 15 weight % to at most 25 weight %, preferably at least 18 weight % to at most 22 weight %, preferably about 20 weight % water; compared to the total weight of the gelatinous shell composition; and,
a first gelatine product with a gel strength of at least 200 g to at most 300 g, preferably at least 205 g to at most 290 g, preferably at least 210 g to at most 285 g, preferably at least 215 g to at most 270 g, preferably at least 215 g to at most 265 g, preferably at least 215 g to at most 250 g, preferably at least 215 g to at most 240 g, preferably at least 215 g to at most 235 g, the gel strength being determined at 6.67%, 10° C., 17H according to Gelatine Manufacturers of Europe (GME); and,
a first gelatine product having a viscosity of at least 1.0 mPa·s to at most 10.0 mPa·s, preferably at least 2.0 mPa·s to at most 8.0 mPa·s, preferably at least 3.0 mPa·s to at most 6.0 mPa·s, preferably at least 3.2 mPa·s to at most 5.5 mPa·s, preferably at least 3.4 mPa·s to at most 5.0 mPa·s, preferably at least 4.2 mPa·s to at most 5.0 mPa·s, preferably at least 4.4 mPa·s to at most 5.0 mPa·s, preferably at least 4.4 mPa·s to at most 4.7 mPa·s; the viscosity being determined at 6.67%, 60° C. according to the method described by the Gelatine Manufacturers of Europe (GME).

Especially this combination of low amount of water and gelatine with a high gel strength results in a fast gelation of the gelatinous shell composition. A fast gelation of the gelatinous shell composition is beneficial as it prevents mixing of the gelatinous shell composition and the filling composition, or the movement of the filling composition after depositing.

In some embodiments, the filling composition is a water-based composition. This allows for water soluble active ingredient to be formulated in the seamless centre filled gummies.

In some embodiments, the filling composition comprises at most 50 weight %, preferably at most 40 weight %, preferably at most 30 weight %, preferably at most 25 weight %, preferably at most 20 weight %, preferably at most 15 weight % water, preferably at most 10 weight % water; compared to the total weight of the filling composition. Such low amount of water does not require or require only very limited amount of water to leave the composition, for the composition set or gelate. However, as the filling may remain liquid, higher amounts of water can be present in the filling composition than in the gelatinous shell composition.

In some embodiments, the filling composition is an oil-based composition.

In some embodiments, the filling composition may comprise a second gelatine product. In some embodiments, the second gelatine product is the same as the first gelatine product.

In some embodiments, the first gelatine product and/or the second gelatine product are bovine, chicken, fish or porcine extracts, preferably bovine extracts from bone, skin, or hide

In some embodiments, the first gelatine product and/or the second gelatine product are acid extracted or alkaline extracted, preferably alkaline extracted.

In some embodiments, the first gelatine product and the second gelatine product are the same.

In some embodiments, the gelatinous shell composition may comprise a sweetener. In some embodiments, the filling composition may comprise a sweetener. Preferably the sweetener is independently selected from the list comprising sugar, honey, sugar syrup, polyol, polysaccharides, . . . . Preferably the sugar is glucose or sucrose.

In some embodiments, the gelatinous shell composition may comprise one or more a secondary ingredients. In some embodiments, the filling composition may comprise one or more a secondary ingredients. Preferably the secondary ingredient may be selected from the list comprising a food acid, such as citric acid, a colorant, flavouring, a third gelatine product, a hydrocolloid, an emulsifier, a preservative, UV-absorbing agent and/or an opacifying agent.

In some embodiments, the gelatinous shell composition may comprise one or more active ingredients. This allows to formulate two or more active ingredients in one gummy, i.e. one (or more) active ingredients in the gelatinous shell and one (or more) active ingredient in the filling. This would allow separating two active ingredients in one gummy, who otherwise would negatively interact with each other.

In some embodiments, the gelatinous shell composition and the filling composition are deposited in a one-shot deposition method.

In some embodiments, the setting of the deposited gelatinous shell composition is performed at least 1° C. to at most 35° C., preferably at least 3° C. to at most 30° C., preferably at least 5° C. to at most 25° C., preferably at least 7° C. to at most 20° C., preferably at least 10° C. to at most 15° C.

In some embodiments, the setting of the deposited gelatinous shell composition is performed at most 35° C., preferably at most 30° C., preferably at most 25° C., preferably at most 20° C., preferably at most 15° C., preferably at most 10° C., preferably at most 7° C., preferably at most 5° C.

In some embodiments, the setting of the deposited gelatinous shell composition sets in at most 120 minutes, preferably at most 90 minutes, preferably at most 60 minutes, preferably at most 45 minutes, preferably at most 30 minutes, preferably at most 20 minutes, preferably at most 15 minutes, preferably at most 10 minutes.

In some embodiments, the gelatinous shell composition comprises at least 5 weight % to at most 15 weight %, preferably at least 6 weight % to at most 12 weight %, at least 7 weight % to at most 10 weight %, at least 7 weight % to at most 9 weight % first gelatine composition, compared to the total weight of the gelatinous shell composition.

In some embodiments, the gelatinous shell composition comprises at least 10 weight % to at most 90 weight %, preferably at least 20 weight % to at most 85 weight %, at least 30 weight % to at most 80 weight %, at least 40 weight % to at most 75 weight %, at least 50 weight % to at most 70 weight % sweetener, compared to the total weight of the gelatinous shell composition.

In some embodiments, the gelatinous shell composition comprises at most 1.0 weight %, preferably at least 0.1 weight % to at most 0.7 weight %, at least 0.2 weight % to at most 0.5 weight %, at least 0.3 weight % to at most 0.4 weight % pectin, compared to the total weight of the gelatinous shell composition.

In some embodiments, the gelatinous shell composition comprises at least 0.5 weight % to at most 5 weight % pH modulating agent, compared to the total weight of the gelatinous shell composition. Preferably the pH modulating agent comprises an acid, preferably citric acid.

In some embodiments, the pH of the gelatinous shell composition is at least 2.8 to at most 3.5, preferably at least 3.0 to at most 3.2.

In some embodiments, the dry weight of the gelatinous shell composition is at least 70 weight % to at most 90 weight %, preferably at least 75 weight % to at most 85 weight %, preferably at least 78 weight % to at most 82 weight %, preferably about 80 weight %, compared to the total weight of the gelatinous shell composition. In some embodiments, the dry weight of the filling composition is at least 50 weight % to at most 90 weight %, preferably at least 60 weight % to at most 85 weight %, at least 70 weight % to at most 80 weight %, about 80 weight %, compared to the total weight of the filling composition.

In some embodiments, the method comprises the step of demoulding the seamless centre filled gummy from the mould.

The invention provides in a seamless centre filled gummy, produced by a method according to embodiment described herein.

The invention provides in a seamless centre filled gummy comprising:
a gelatinous shell; and,
a filling encased inside said gelatinous shell;
wherein the gelatinous shell comprises:
a first gelatine product;
optionally at least one first sweetener; and,
optionally one or more secondary ingredients;
wherein the filling comprises at least one active ingredient and at least one ingredient selected from the following:
   a second gelatine product;
   food oil, such as coconut oil
   emulsified food oil;
   collagen peptides or hydrolyzed collagen peptides;
   soluble fibres, such as inulin or polydextrose;
   maltodextrin or dextrine;
   glycerol; or,
   polyethylene glycol.

In some embodiments, the filling of the seamless centre filled gummy is at 22° C. liquid, pasty or solid, preferably liquid or pasty, more preferably liquid.

In some embodiments, the first gelatine product and/or optionally the second gelatine product have a gel strength of at least 200 g to at most 300 g, preferably at least 205 g to at most 290 g, preferably at least 210 g to at most 285 g, preferably at least 215 g to at most 265 g, preferably at least 215 g to at most 240 g, preferably at least 215 g to at most 235 g, the gel strength being determined at 6.67%, 10° C., 17H according to the method described by the Gelatine Manufacturers of Europe (GME). Such high gel strengths may make the gummy chewable, yet easy to swallow.

In some embodiments, the first gelatine product and/or optionally the second gelatine product have a viscosity of at least 1.0 mPa·s to at most 10.0 mPa·s, preferably at least 2.0 mPa·s to at most 8.0 mPa·s, preferably at least at least 3.0 mPa·s to at most 6.0 mPa·s, preferably at least at least 3.2 mPa·s to at most 5.5 mPa·s, preferably at least 3.4 mPa·s to at most 5.0 mPa·s, preferably at least 4.2 mPa·s to at most 5.0 mPa·s, preferably at least 4.4 mPa·s to at most 5.0 mPa·s, preferably at least 4.4 mPa·s to at most 4.7 mPa·s the viscosity being determined at 6.67%, 60° C. according to the method described by the Gelatine Manufacturers of Europe (GME).

In some embodiments, the seamless centre filled gummy comprises at least 5 weight % to at most 40 weight %, preferably at least 10 weight % to at most 30 weight %, preferably at least 15 weight % to at most 25 weight %, preferably at least 17 weight % to at most 22 weight % filling.

In some embodiments, the seamless centre filled gummy comprises at least 60 weight % to at most 95 weight %, preferably at least 70 weight % to at most 90 weight %, preferably at least 75 weight % to at most 85 weight %, preferably at least 78 weight % to at most 83 weight % gelatinous shell.

### EXAMPLES

Methods

Unless otherwise indicated, the viscosity of compositions at a certain temperature is measured
with Brookfield LVDV2T viscosimeter equipped with LV3 spindle;
180 ml of composition (at selected temperature) is poured in a plastic cup (diameter 55 mm, height 95 mm);
Spindle is attached to the viscometer and lowered into the composition;
Rotation speed of the spindle is set so the torque is within 10-90% of the measuring range;
Viscosity is read from the Brookfield after 5 min;
During analysis, the composition is kept at the selected temperature using a cryostate & double walled glass vessel.

Unless otherwise indicated, the absolute density of compositions at a certain temperature is measured with a pycnometer, preferably 50 ml.

Gelatin

In the following examples, an alkaline process gelatine extracted from bovine bones for edible applications is used, with the following parameters:

| | | Test |
|---|---|---|
| Standard parameters | Specifications | Method |
| Gel strength (6.67%, | 215-235 g | GME |
| 10° C., 17 H) | | |
| Viscosity (6.67%, 60° C.) | 4.4-5.0 mPa · s | GME |
| pH (6.67%, 45° C.) | 5.3-6.2 | |
| Loss on drying | ≤13% | GME |
| Residue on ignition | ≤2.0% | GME |

### Example 1: Centre Filled Gummy, Comprising Vitamin C

For the preparation of the gelatinous shell composition/composition the following ingredient were used:

Gelatinous shell composition

| | |
|---|---|
| Gelatine | 8 kg |
| Hot water | 15 kg |
| Sugar (sucrose) | 43.2 kg |
| Glucose syrup 60DE | 36 kg |
| Water | 12 kg |
| Citric acid (50% solution) | 2.6 kg |
| Flavour, colour | q.s. |

The following procedure was used to produce the gelatinous shell composition:
The gelatine was dissolved in hot water (80-90° C.), to obtain a first gelatine solution.
The sucrose, glucose syrup and water were combined and boiled to 125° C. the obtained solution was cooled below 100° c. and the combined with the first gelatine solution, the citric acid and the flavour and colour.
The obtained gelatinous shell composition has a water content of 20 weight %, due to boiling.

For the preparation of the filling composition/composition the following ingredient were used:

Filling composition

| | |
|---|---|
| Gelatine | 1.0 kg |
| Water | 16.7 kg |
| Sodium Ascorbate | 15 kg |
| Glucose syrup 40DE | 67.3 kg |

The following procedure was used to produce the filling composition:
The gelatine was dissolved in hot water (50° C.), to obtain a second gelatine solution.
The second gelatine solution was cooled to below 35° C.
Sodium Ascorbate was added to the cooled second gelatine solution, and stirred until fully dissolved.
The glucose syrup was mixed in the above obtained solution at 25° C., to form the filling composition.

Depositing:

The above obtained gelatinous shell composition at 60° C. and the filling composition at 25° C. were deposited in a mould or blister using a centre-filling, one shot depositing system from Baker Perkins. During each depositing, 1.9 g gelatinous shell composition and 0.6 g filling composition was used. A phase offset was used between the depositing of the gelatinous shell composition and the depositing of the filling composition, so that the filling composition was completely encased by the gelatinous shell composition.

The above obtained gelatinous shell composition has 60° C. at an absolute density of 1.36 g/cm3 and a viscosity of 8000 mPa·s, and is deposited from t₁=0 seconds to t₄=0.60 seconds.

The above obtained filling composition has 25° C. at an absolute density of 1.38 g/cm3 and a viscosity of 6800 mPa·s, and is deposited from t₂=0.16 seconds to t₃=0.40 seconds.

The filled moulds were left at ambient temperature (20° C.) under ventilation for 8 to 30 minutes to set. Seamless centre filled gummies were obtained after demoulding.

### Example 2: Centre Filled Gummy, Comprising Acacia Honey, Propolis Extract and Vitamin C, which have a Positive Effect on Throat Health

For the preparation of the gelatinous shell composition the following ingredient were used:

Gelatinous shell composition

| | |
|---|---|
| Gelatine | 8 kg |
| Hot water | 15 kg |
| Sugar (sucrose) | 43.2 kg |
| Glucose syrup 60DE | 36 kg |
| Water | 12 kg |
| Citric acid (50% solution) | 2.6 kg |
| Flavour, colour | q.s. |

The following procedure was used to produce the gelatinous shell composition:
The gelatine was dissolved in hot water (80-90° C.), to obtain a first gelatine solution.
The sucrose, glucose syrup and water were combined and boiled to 125° C. the obtained solution was cooled below 100° c. and the combined with the first gelatine solution, the citric acid and the flavour and colour.
The obtained gelatinous shell composition has a water content of 20 weight %, due to boiling.

For the preparation of the filling composition the following ingredient were used:

Filling composition

| | |
|---|---|
| Gelatine | 0.2 kg |
| Water | 3.4 kg |
| Sodium Ascorbate | 3.07 kg |
| Glucose syrup 40DE | 44.58 kg |
| Acacia honey | 44.58 kg |
| Propolis Intens Extract | 4.17 kg |

The following procedure was used to produce the filling composition:
The gelatine was dissolved in hot water (50° C.), to obtain a second gelatine solution.
The second gelatine solution was cooled to below 35° C.
Sodium Ascorbate was added to the cooled second gelatine solution, and stirred until fully dissolved.
The glucose syrup, the Acacia honey and the propolis intens extract were mixed in the above obtained solution at 25° C., to form the filling composition.

Depositing:

The above obtained gelatinous shell composition at 60° C. and the filling composition at 25° C. were deposited in a mould or blister using a centre-filling, one shot depositing system from Baker Perkins. During each depositing, 1.9 g gelatinous shell composition and 0.6 g filling composition was used. A phase offset was used between the depositing of the gelatinous shell composition and the depositing of the filling composition, so that the filling composition was completely encased by the gelatinous shell composition.

The above obtained gelatinous shell composition has 60° C. at an absolute density of 1.36 g/cm3 and a viscosity of 8000 mPa·s, and is deposited from t₁=0 seconds to t₄=0.60 seconds.

The above obtained filling composition has 25° C. at an absolute density of 1.37 g/cm3 and a viscosity of 4700 mPa·s, and is deposited from t₂=0.16 seconds to t₃=0.40 seconds.

The filled moulds were left at ambient temperature (20° C.) under ventilation for 8 to 30 minutes to set. Seamless centre filled gummies were obtained after demoulding.

### Example 3: Centre Filled Gummy, Comprising Ibuprofen, which has an Inflammatory Effect

For the preparation of the gelatinous shell composition the following ingredient were used:

Gelatinous shell composition

| | |
|---|---|
| Gelatine | 8 kg |
| Hot water | 15 kg |
| Sugar (sucrose) | 43.2 kg |
| Glucose syrup 60DE | 36 kg |
| Water | 12 kg |
| Citric acid (50% solution) | 2.6 kg |
| Flavour, colour | q.s. |

The following procedure was used to produce the gelatinous shell composition:
The gelatine was dissolved in hot water (80-90° C.), to obtain a first gelatine solution.
The sucrose, glucose syrup and water were combined and boiled to 125° C. the obtained solution was cooled below 100° c. and the combined with the first gelatine solution, the citric acid and the flavour and colour.
The obtained gelatinous shell composition has a water content of 20 weight %, due to boiling

For the preparation of the filling composition the following ingredient were used:

Filling composition

| | |
|---|---|
| Sodium Ibuprofen | 9.46 kg |
| Water | 2.1 kg |
| Ethanol | 2.1 kg |
| Glucose syrup 40DE | 86.34 kg |

The following procedure was used to produce the filling composition:
Sodium Ibuprofen was dissolved in a mixture of water and ethanol at 25° C.
The glucose syrup was heated to 35° C. and mixed in the above obtained solution, to form the filling composition.

Deposition:

The above obtained gelatinous shell composition at 60° C. and the filling composition at 35° C. were deposited in a mould or blister using a centre-filling, one shot depositing system from Baker Perkins. For each deposition, 1.9 g gelatinous shell composition and 0.6 g filling composition was used. A phase offset was used between the depositing of the gelatinous shell composition and the depositing of the filling composition, so that the filling composition was completely encased by the gelatinous shell composition. The above obtained gelatinous shell composition has at 60° C. an absolute density of 1.36 g/cm³ and a viscosity of 8000 mPa·s, and is deposited from t1=0 seconds to t4=0.60 seconds.

The above mentioned filling composition has at 35° C. an absolute density of 1.35 g/cm³ and a viscosity of 7000 mPa·s, and is deposited from t2=0.16 seconds to t3=0.40 seconds.

The filled moulds were left at ambient temperature (20° C.) under ventilation for 8 to 30 minutes to set.

### Example 4: Centre Filled Gummy, Comprising Iron which has a Positive Effect on Immunity

For the preparation of the gelatinous shell composition the following ingredient were used:

Gelatinous shell composition

| | |
|---|---|
| Gelatine | 8 kg |
| Hot water | 15 kg |
| Sugar (sucrose) | 43.2 kg |
| Glucose syrup 60DE | 36 kg |
| Water | 12 kg |
| Citric acid (50% solution) | 2.6 kg |
| Flavour, colour | q.s. |

The following procedure was used to produce the gelatinous shell composition:
The gelatine was dissolved in hot water (80-90° C.), to obtain a first gelatine solution.
The sucrose, glucose syrup and water were combined and boiled to 125° C. the obtained solution was cooled below 100° c. and the combined with the first gelatine solution, the citric acid and the flavour and colour.
The obtained gelatinous shell composition has a water content of 20 weight %, due to boiling

For the preparation of the filling composition the following ingredient were used:

Filling composition

| | |
|---|---|
| Lipofer ™ microcapsules | 7.27 kg |
| Water | 9.33 kg |
| Glucose syrup 60DE | 41.7 kg |
| Glucose syrup 40DE | 41.7 kg |

The following procedure was used to produce the filling composition:
Lipofer™ microcapsules was dissolved in water at 25° C.
The glucose syrup was mixed in the above obtained solution, to form the filling composition.

Deposition:

The above obtained gelatinous shell composition at 60° C. and the filling composition at 25° C. were deposited in a mould or blister using a centre-filling, one shot depositing system from Baker Perkins. For each deposition, 1.9 g gelatinous shell composition and 0.6 g filling composition was used. A phase offset was used between the depositing of the gelatinous shell composition and the depositing of the filling composition, so that the filling composition was completely encased by the gelatinous shell composition. The above obtained gelatinous shell composition has at 60° C. an absolute density of 1.36 g/cm³ and a viscosity of 8000 mPa·s, and is deposited from t1=0 seconds to t4=0.60 seconds.

The above mentioned filling composition has at 25° C. an absolute density of 1.37 g/cm³ and a viscosity of 5700 mPa·s, and is deposited from t2=0.16 seconds to t3=0.40 seconds.

The filled moulds were left at ambient temperature (20° C.) under ventilation for 8 to 30 minutes to set.

1. A method for producing a seamless centre filled gummy, said seamless centre filled gummy comprising a gelatinous shell and a filling encased inside said gelatinous shell; wherein the filling comprises at least one active ingredient;
the method comprising the steps of:
1) preparing a gelatinous shell composition by:
1a) dissolving a first gelatine product in water to form a first gelatine solution;
1b) optionally adding at least one first sweetener and optionally one or more secondary ingredients to the first gelatine solution;
2) preparing a filling composition, wherein the filling composition comprises at least one bulking agent and one or more active ingredients dissolved or suspended in said filling composition;
3) depositing the gelatinous shell composition and the filling composition within the same timeframe;
thereby fully encasing the filling composition with the gelatinous shell composition;
4) setting the deposited gelatinous shell composition;
wherein the temperature Tᵢₙₙₑᵣ of the filling composition upon deposition is at most 45° C., and wherein the viscosity of the filling composition upon deposition ranges between 500 and 30000 mPa·s.

2. The method according to claim 1, wherein said one or more active ingredients are selected from the list comprising vitamins, minerals, probiotics, herbal extracts, essential fatty acids, hydrolyzed collagen peptides, pharmaceuticals or a combination thereof.

3. The method according to claim 1 or 2, wherein the temperature Tₒᵤₜₑᵣ of the gelatinous shell composition upon deposition is at least at least 40° C. to at most 100° C., preferably at least 45° C. to at most 85° C. preferably at least 50° C. to at most 75° C., preferably at least 55° C. to at most 65° C., preferably 60° C.; and/or wherein the temperature Tᵢₙₙₑᵣ of the filling composition upon deposition is at most 40° C., preferably at most 35° C., preferably at most 30° C., preferably at most 25° C., preferably at most 23° C., preferably at most 20° C., preferably at most 15° C., preferably at most 10° C., preferably at most 7° C., preferably at most 5° C.

4. The method according to any one of claims 1 to 3, wherein the viscosity of the gelatinous shell composition at temperature Tₒᵤₜₑᵣ is at least 500 mPa·s to at most 30000 mPa·s, preferably at least 1000 mPa·s to at most 25000 mPa·s, preferably at least 1500 mPa·s to at most 20000 mPa·s, preferably at least 2000 mPa·s to at most 17000 mPa·s, preferably at least 2500 mPa·s to at most 15000 mPa·s, preferably at least 3000 mPa·s to at most 12000 mPa·s; and/or,
wherein the viscosity of the filling composition at temperature Tᵢₙₙₑᵣ is at least 1000 mPa·s to at most 25000 mPa·s, preferably at least 1500 mPa·s to at most 20000 mPa·s, preferably at least 2000 mPa·s to at most 17000 mPa·s, preferably at least 2500 mPa·s to at most 15000 mPa·s, preferably at least 3000 mPa·s to at most 12000 mPa·s.

5. The method according to any one of claims 1 to 4, wherein the absolute density of the gelatinous shell composition at temperature Tₒᵤₜₑᵣ is at least 1.25 g/cm³ to at most 1.50 g/cm³, preferably at least 1.30 g/cm³ to at most 1.45 g/cm³, preferably at least 1.32 g/cm³ to at most 1.40 g/cm³, preferably at least 1.35 g/cm³ to at most 1.38 g/cm³; and/or,
wherein the absolute density of the filling composition at temperature Tᵢₙₙₑᵣ is at least 1.25 g/cm³ to at most 1.50 g/cm³, preferably at least 1.30 g/cm³ to at most 1.45 g/cm³, preferably at least 1.32 g/cm³ to at most 1.40 g/cm³, preferably at least 1.35 g/cm³ to at most 1.38 g/cm³.

6. The method according to any one of claims 1 to 5, wherein the first gelatine product has a gel strength of at least 200 g to at most 300 g, preferably at least 205 g to at most 290 g, preferably at least 210 g to at most 285 g, preferably at least 210 g to at most 270 g, preferably at least 215 g to at most 265 g, preferably at least 215 g to at most 250 g, preferably at least 215 g to at most 240 g, preferably at least 215 g to at most 235 g, the gel strength being determined at 6.67%, 10° C., 17H according to the method described by the Gelatine Manufacturers of Europe (GME).

7. The method according to any one of claims 1 to 6, wherein the first gelatine product has a viscosity of at least 1.0 mPa·s to at most 10.0 mPa·s, preferably at least 2.0 mPa·s to at most 8.0 mPa·s, preferably at least at least 3.0 mPa·s to at most 6.0 mPa·s, preferably at least at least 3.2 mPa·s to at most 5.5 mPa·s, preferably at least 3.4 mPa·s to at most 5.0 mPa·s, preferably at least 4.2 mPa·s to at most 5.0 mPa·s, preferably at least 4.4 mPa·s to at most 5.0 mPa·s, preferably at least 4.4 mPa·s to at most 4.7 mPa·s the viscosity being determined at 6.67%, 60° C. according to the method described by the Gelatine Manufacturers of Europe (GME).

8. The method according to any one of claims 1 to 7, wherein the first gelatine product has a pH of at least 4.0 to at most 7.0, preferably at least 4.5 to at most 6.5, preferably at least at least 4.5 to at most 6.2, preferably at least at least 5.0 to at most 6.2, preferably at least 5.3 to at most 6.2, preferably at least 5.3 to at most 6.0, the pH being determined at 6.67%, 45° C. according to the method described by the Gelatine Manufacturers of Europe (GME).

9. The method according to any one of claims 1 to 8, wherein the gelatinous shell composition and the filling composition are deposited in a non-starch mould, preferably a plastic mould, silicon mould, metal mould or a blister.

10. The method according to any one of claims 1 to 9, wherein the bulking agent is selected from the list comprising:
a second gelatine product;
food oil, such as coconut oil;
emulsified food oil;
collagen peptides or hydrolyzed collagen peptides;
soluble fibers, such as inulin, polydextrose;
carbohydrate polymers; such as dextrin, maltodextrin;
glycerol; or,
polyethylene glycol.

11. The method according to any one of claims 1 to 10, wherein the gelatinous shell composition comprises at least 10 weight % to at most 30 weight %, preferably at least 15 weight % to at most 25 weight %, preferably at least 18 weight % to at most 22 weight %, preferably about 20 weight % water; compared to the total weight of the gelatinous shell composition.

12. A seamless centre filled gummy, produced by a method according to any one of claims 1 to 11.

13. A seamless centre filled gummy comprising:
a gelatinous shell; and,
a filling encased inside said gelatinous shell;
wherein the gelatinous shell comprises:
a first gelatine product;
optionally at least one first sweetener; and,
optionally one or more secondary ingredients;
wherein the filling comprises at least one active ingredient and at least one ingredient selected from the following:
a second gelatine product;
food oil, such as coconut oil
emulsified food oil;
collagen peptides or hydrolyzed collagen peptides;
soluble fibers, such as inulin or polydextrose;
maltodextrin or dextrine;
glycerol; or,
polyethylene glycol.

14. The seamless centre filled gummy according to claim 12 or 13, wherein the first gelatine product and/or optionally the second gelatine product have a gel strength of at least 200 g to at most 300 g, preferably at least 205 g to at most 290 g, preferably at least 210 g to at most 285 g, preferably at least 210 g to at most 270 g, preferably at least 215 g to at most 265 g, preferably at least 215 g to at most 250 g, preferably at least 215 g to at most 240 g, preferably at least 215 g to at most 235 g, the gel strength being determined at 6.67%, 10° C., 17H according to the method described by the Gelatine Manufacturers of Europe (GME).

15. The seamless centre filled gummy according to any one of claims 12 to 14, wherein the first gelatine product and/or optionally the second gelatine product have a viscosity of at least 1.0 mPa·s to at most 10.0 mPa·s, preferably at least 2.0 mPa·s to at most 8.0 mPa·s, preferably at least at least 3.0 mPa·s to at most 6.0 mPa·s, preferably at least at least 3.2 mPa·s to at most 5.5 mPa·s, preferably at least 3.4 mPa·s to at most 5.0 mPa·s, preferably at least 4.2 mPa·s to at most 5.0 mPa·s, preferably at least 4.4 mPa·s to at most 5.0 mPa·s, preferably at least 4.4 mPa·s to at most 4.7 mPa·s the viscosity being determined at 6.67%, 60° C. according to Gelatine Manufacturers of Europe (GME).
